(19) United States
(12) Patent Application Publication
(10) Pub. No.: US 2005/0226921 A1
(43) Pub. Date: Oct. 13, 2005

(54) Non-invasive analysis and controlled dosage transdermal active patch

(76) Inventor: Robert N. Kortzebom, San Mateo, CA (US)

Correspondence Address: Harris Zimmerman;Law Offices of Harris Zimmerman, Suite 710, 1330 Broadway, Oakland, CA, 94612, US

(21) Appl. No.: 10/728,660

(22) Filed: Apr. 13, 2004

(51) Int. Cl. A61K 9/70; A61N 1/30

(52) U.S. Cl. 424/449; 604/20

(57) ABSTRACT

A programmable transdermal patch non-invasively delivers pharmaceuticals or other bio-active agents through the skin of a living body. The patch contains one or more agent storage pads and one or more active drivers that apply an electric current to the skin or produce ultrasound to drive the agent into the skin. A digital data processor controls the drivers to match administration of the agents to the needs of the body. The patch may contain a sensor, coupled to the data processor, for monitoring the concentration of a substance in the body in order to vary dosage of a therapeutic agent. A radio contained in the patch enables control by medical personnel from a remote location and/or transmission of sensor data to the remote location. The pads, drivers, sensor, data processor, radio and a battery are all contained within a unitary patch and need no physical connection to external devices.

### CROSS-REFERENCE TO RELATED APPLICATION

(Not Applicable)

### STATEMENT REGARDING FEDERALLY SPONSORED RESEARCH OR DEVELOPMENT

(Not Applicable)

### BACKGROUND OF THE INVENTION

This invention relates to transdermal patches for delivering bio-active agents through the skin of a living body and to apparatus for controlling the rate and timing of transdermal delivery of medicinal drugs or other bio-active agents through the skin.

Non invasive transdermal delivery has been used to administer a variety of different drugs, examples of which include nicotine to assist persons in stopping smoking, estrogen for hormone therapy, nitroglycerin for angina, scopolamine for motion sickness, fentanyl for pain control, clonidine for hypertension and ethinylestradiol and norelgestromin for contraception purposes.

The conventional transdermal patch contains an adhesive pad which is fastened to the skin and which serves as a permeable reservoir containing a drug which is to be administered. Molecules of the drug pass through minute gaps between skin cells and through the skin's pores. Patches of this kind have a number of advantages over other methods of administering drugs or other agents. The process is non-invasive. It does not require physical penetration of the skin as in the case of hypodermic injections or intravenous administration of drugs. It bypasses the digestive and other metabolic processes which can alter and consume drugs which are ingested orally. In its original and simplest forms the patch can be small and flat and needs no connections to external control devices, drug containers or the like. Thus the patch can be inconspicuous and does not restrict the mobility of the wearer.

Other characteristics limit usage of the original forms of transdermal patch to administration of only a small number of drugs. For example, diffusion of the drug out of the reservoir and into the skin is a passive process relying only on a concentration gradient. The stratum corneum or outer layer of the skin forms a barrier of dead cells which can adversely affect the rate at which substances pass through the skin by unaided diffusion. Drug molecules must be small enough to pass between the cells in order to reach capillaries deeper in the skin. The stratum corneum varies in thickness and porosity from person to person, so the drug should have a broad range of acceptable concentrations. Only a small number of drugs have characteristics which enable un-aided diffusion through the stratum corneum at an adequate rate.

The rate of dosage by the above described original transdermal patches is not adjustable and falls off over a period of use as the concentration of the drug in the reservoir pad diminishes. The rate at which drug is released is dependent on the composition of the reservoir pad, on characteristics of the particular drug and on properties of the area of skin to which it is applied. Designing a conventional patch of this kind to maintain a desired concentration of a particular drug in the body can be very exacting and in many cases is not practical. Further, the conventional patch does not enable any programmed variation of dosage rate over a period of time and dosage cannot be adjusted by medical personnel after the patch is in place. This is of particular significance in the case of administration of certain drugs of which the administration of insulin to diabetic patients is one example. A patient's need for insulin depends on the current concentration of glucose in the body and this may vary in an unpredictable manner during a period of time. Traditionally, diabetic patients have been required to prick their skin periodically in the course of a day, perform an analysis of the glucose concentration in a drop of blood and to self administer insulin if needed. This is a painful and sometimes unreliable procedure. The above described characteristics of the original transdermal patches make them unsuitable for administering insulin or other drugs which are subject to a variable dosage requirement.

More recent advances in transdermal drug delivery address the problems discussed above. Delivery of bio-active agents through the skin has been enhanced by active driving processes which enable drugs of larger molecular size to be administered and which provide for control of the rate of drug delivery. In one such process, known as iontophoresis, electrodes are used to transmit a small electric current through the reservoir pad and into the underlying skin. The current is thought to temporarily enlarge porosities in the stratum corneum. Drugs dissolved in the reservoir pad tend to be ionized and the electrical field impels the charged ions through the enlarged porosities. Diffusion of agents into the skin has also been actively controlled by another driving process, known as phonophoresis, in which ultrasound is used to increase the porosity of the stratum corneum.

Some more recent transdermal drug delivery systems also make use of a digital data processor to control the action of the active drivers. This enables programmable variation of the timing and rate of drug delivery to accommodate to different drugs and to the needs of different patients.

Sensors which monitor the concentration of a substance in a patient's body in a non-invasive manner have been coupled to the digital data processor. Such sensors typically employ a process known as reverse iontophoresis. Electrodes produce an electrical current in the skin which extracts interstitial fluid, including glucose for example, through the skin. Glucose concentration in the interstitial fluid is detected by infrared spectography for example. This enables computer controlled variation of insulin dosage to match the needs of the particular patient.

These recent advances have greatly expanded the versatility and effectiveness of transdermal drug delivery but have also created problems which can restrict usage of the technique. Instead of a single unitary patch, the newer systems variously require that multiple components be fastened to the skin, require interconnecting cables and/or require bulky external housings containing controls or other components. Operation may require the presence of medical personnel or may be dependent on actions taken by the patient. Unlike the original and simpler transdermal patches, these drug delivery systems are not free of physical connections to external devices and are not fully mobile.

The present invention is directed to overcoming one or more of the problems discussed above.

### BRIEF SUMMARY OF THE INVENTION

In one aspect the present invention provides a transdermal patch for delivery of a bio-active agent into the skin of a living body which patch is fastenable to a surface of the skin. The patch contains at least one agent storage pad positioned to dispense agent into the skin and contains electrically operated driver means for causing delivery of the stored agent from the storage pad into the skin. A battery supplies electrical current to the driver means and other electrical components of the patch. A programmable digital data processor controls dispensing of the agent by the reservoir pad and driver means. An analysis unit monitors the concentration of a substance in the body. The analysis unit provides concentration signals to the digital data processor enabling dispensing of the agent into the skin when the concentration is outside of a particular range of concentrations. The battery, programmable digital data processor and the analysis unit are all contained within the patch itself.

In another aspect of the invention, the patch may contain a plurality of the agent storage pads, each storing a different agent, and a plurality of the electrically operated driver means each being operative on a separate one of the agent storage pads in response to actuating signals from the data processor.

In still another aspect, the invention provides a transdermal patch for delivery of a bio-active agent into the skin of a living body which patch is fastenable to a surface of the skin. The patch contains an agent storage pad positioned to dispense agent into the skin and contains electrically operated driver means for causing delivery of the stored agent from the storage pad into the skin. A battery supplies electrical current to the driver means and other electrical components of the patch. A programmable digital data processor controls dispensing of the agent by the reservoir pad and driver means and a radio receiver enables input of programming signals to the data processor from a remote location. The programmable digital data processor, radio receiver and battery are contained within the patch.

The invention provides an “intelligent” transdermal patch which regulates release of pharmaceuticals or other bio-active agents into the body to establish and maintain a preferred dosage over a period of time. Administration of the bio-active agent through the skin is controlled by application of an electrical current or application of ultrasound to one or more agent storage pads. A digital data processor chip contained within the patch may variously be programmed to match the administration of the agent to a known rate at which the agent is consumed by metabolic processes or may respond to a sensor in the patch which monitors the concentration of a substance in the body. In some usages of the invention, the data processor is programmed to vary the rate of release of the bio-active agent to conform to normal variations of the rate at which hormones or other substances are produced by the body during the course of a day or other time period. The patch may contain a radio receiver for delivering programming signals, originating at a remote radio transmitter, to the data processor. This enables control of the patch by medical personnel or other persons from a location which is away from the patch. The patch may contain a plurality of agent storage pads each holding a different bio-active agent which agents may be released jointly or independently of each other as might be needed. The patch may be used to administer diverse different pharmaceuticals, vaccines or other bio-active substances without significant pain or inconvenience to the person wearing the patch and without requiring the wearer to self regulate dosage of the bio-active substance. The unitary patch requires no physical connection to external devices and thus allows the wearer to be fully mobile.

The invention, together with further objects and advantages thereof, may be further understood by reference to the following detailed description of the invention and by reference to the accompanying drawings.

### BRIEF DESCRIPTION OF THE SEVERAL VIEWS OF THE DRAWINGS

In the accompanying drawings:

FIG. 1 is a broken out side view of a controlled dosage transdermal patch depicting a first embodiment of the invention which enables controlled administration of any of a plurality of different bio-active agents.

FIG. 2 is a broken out view of the underside or skin facing surface of the transdermal patch of FIG. 1 taken along line 2-2 of FIG. 1.

FIG. 3 is a is a graph depicting a typical variation of the concentration of a pharmaceutical drug within the body of a medical patient over a period of time during controlled administration of the drug by a transdermal patch embodying the invention.

FIG. 4 is a section view taken along line 4-4 of FIG. 2 and which depicts an analysis unit within the patch which monitors the concentration of substances in interstitial fluid extracted through the skin.

FIG. 5 is a schematic diagram illustrating characteristics of the infrared absorption spectra of substances in extracted interstitial fluid which are detected by the analysis unit of the patch which is shown in FIG. 4.

FIG. 6 is an enlarged view of a corner region of the transdermal patch of the preceding figures depicting a patch activating switch.

FIG. 7 is a section view taken along line 7-7 of FIG. 6 showing internal components of the switch in the open condition.

FIG. 8 is a section view of the activating switch of FIG. 7 showing components of the switch in the closed condition.

FIG. 9 is a broken out view of a modification of a portion of the transdermal patch of the preceding figures wherein administration of the drug is controlled by a membrane which is permeable when subjected to an electrical current and impermeable in the absence of the current.

FIG. 10 is a broken out view of the underside of an embodiment of the transdermal patch in which controlled diffusion of a bio-active agent into the skin is effected by ultrasound generators within the patch.

### DETAILED DESCRIPTION OF THE INVENTION

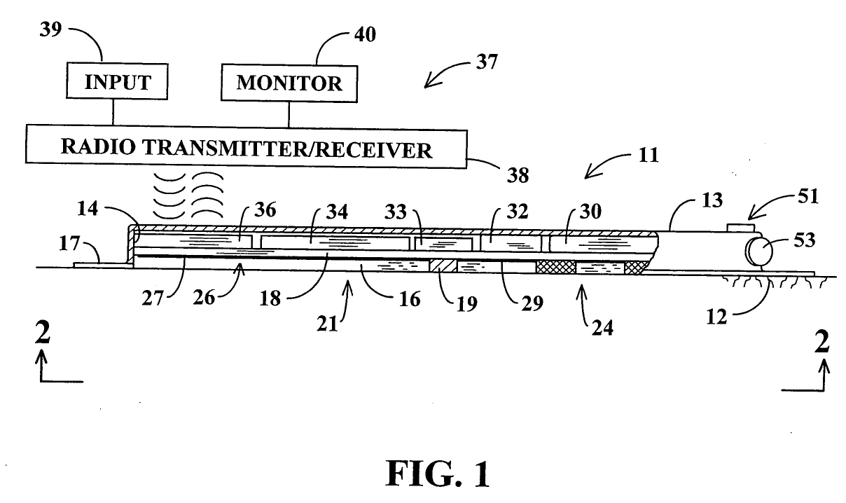

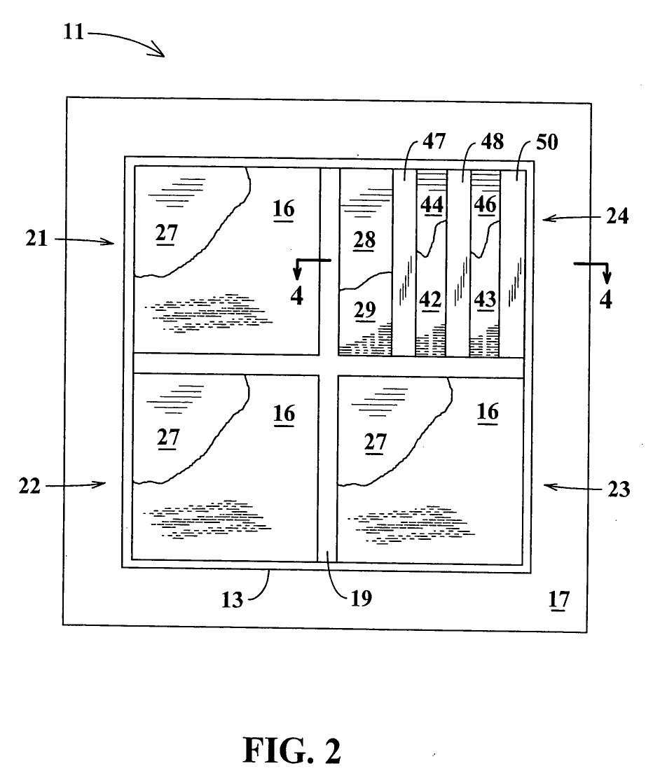

Referring jointly to FIGS. 1 and 2 of the drawings, a controlled dosage transdermal patch 11 embodying the invention is adhered to the skin 12 of a person who is to be administered one or more pharmaceutical drugs or other bio-active agents. The patch 11 of this example includes an outer cover 13 forming a thin chamber 14 having an open underside that faces the person's skin. Agent storage pads 16 at the underside of the patch 11 may be of any of the known hydrophilic compositions and are preferably hydrogel pads of the type that adhere to the skin. Retention of the patch may be augmented by a skirt 17 of adhesive tape which extends outward from the periphery of cover 13 at the underside of the cover. Chamber 14 is divided into upper and lower regions by a circuit board 18 which supports electronic components, to be hereinafter described, within the upper region of the chamber.

The patch may be designed to administer a single bio-active agent or to administer any selected one or selected ones of a plurality of agents. The patch 11 of this particular example enables administration of three different bio-active agents. Partitioning 19 divides the lower region of chamber 14 into four square sectors 21, 22, 23 and 24. The first three sectors 21, 22 and 23 contain square agent storage pads 16 situated at the lower region of chamber 14 in position to contact the skin 12. Each such pad 16 functions as an agent reservoir and is initially saturated with a bio-active agent that is to be administered by the particular pad. The fourth sector 24 of this embodiment contains an analysis unit 25 which extracts interstitial fluid through the skin 12 and which detects the concentration of a substance in the extracted fluid as will hereinafter be described in more detail.

The stratum corneum or outermost layer of the skin 12 is normally impermeable or semi-impermeable to many bio-active agents, particularly agents having relatively large molecular structures. Consequently, many drugs or other bio-active agents do not diffuse through the outer layer of the skin 12, at least at a medically desirable rate, simply as a result of the concentration gradient between a drug saturated storage pad 16 and the adjacent skin. This originally limited the use of transdermal patches to a small number of drugs or other agents. Electrically operated drivers can make the stratum corneum temporarily more permeable and can actively drive bio-active agents from the pad 16 into underlying tissue. One known form of active driver performs a process known as iontophoresis in which electrodes create a small and painless electrical current in the skin which increases permeability of the stratum corneum. Drugs dissolved in hydrogel pads exhibit an ionic charge and the electrical field created by the energized electrodes actively drives drug ions into porosities in the skin. Another form of active driver, using a process known as phonophoresis, generates acoustic pulses of ultrasound to increase permeability of the stratum corneum.

The transdermal patch 11 depicted in FIGS. 1 and 2 contains active drivers 26 of the iontophoresis type to control dosage of the bio-active agents. A separate first driver electrode 27 is disposed against the upper surface of each storage pad 16 and each such electrode preferably conforms in outline with the underlying pad. A single second driver electrode 28 is spaced apart from each of the first driver electrodes 27 to enable creation of an electrical current within the skin between one or more of the first driver electrodes 27 and the second electrode 28 In this embodiment, the second driver electrode 28 is situated in the fourth sector 24 of chamber 14 against the top surface of another hydrogel pad 29 which assures good electrical contact between the second driver electrode and skin 12.

Administration of the bio-active agent in any of the storage pads 16 is initiated by applying voltage of a first polarity to the one of the first driver electrodes 27 that contacts that pad while applying voltage of opposite polarity to the second driver electrode 28. The electrical field which is created in this manner repels ions having a polarity similar to that at the first driver electrode 27 and thus drives such ions out of pad 16 and into the underlying skin 12. Thus a positive voltage is applied to the first driver electrode 27 if ions of the drug are of a type which exhibits a positive charge and negative voltage is applied if the drug ions are negatively charged. Administration of the drug stops when application of the voltage to the first driver electrode 27 is terminated.

The electrical force necessary to cause a particular drug to be dispensed from a hydrogel material is dependent on electrical characteristics of the molecules of the drug and is proportional to both the viscosity of the hydrogel and the current within the hydrogel. Additives known to those skilled in the art can be added to the hydrogel material of storage pads 16 to fix the viscosity of the material at a value at which a drug is retained in the pad in the absence of electrical current The degree of viscosity which is needed to stabilize a particular drug in this manner can easily be determined by testing. In this condition, the pads 16 may be characterized as being impermeable in the absence of an electrical current while being permeable in the presence of electrical current. The electrical current which is created by an active driver 26 is dependent on the voltages which are applied to the driver electrodes 27 and 28. The particular voltages that are needed to drive a particular amount of a drug out of the pad 16 in a particular time period can also be determined by testing.

Electrical power for operating the driver electrodes 27 and 28 and for operating other electrical components to be hereinafter described is provided by a battery 30. Other electrical components include a voltage regulating module 32 which provides selectable voltages to a switching module 33. Switching module 33 enables application of selected voltages of either polarity to any or all of the driver electrodes 27 and 28. The voltage regulating module 32 and switching module 33 may be solid state circuits which are controlled by digital signals produced by a programmable digital data processor 34. Data processor 34 is a semiconductor microchip of one of the known forms and includes the standard computer components such as a central processing unit, memory arrays, data buses and input/output interfacing. Data processor 34 is programmable to control the timing and duration of successive administrations of bio-active agent at any of the pads 16 in any of a variety of modes of operation which will hereinafter be discussed. Battery 30, voltage regulating module 32, switching module 33 and data processor 34 are all contained within the transdermal patch 11 on circuit board 18 within the upper region of chamber 14.

The patch 11 also contains a radio transmitter and receiver 36 which enables input of instructions to data processor 34 and monitoring of data produced by the processor with a remote control unit 37 which may be located away from the patch. The remote control unit 37 in this embodiment includes another radio transmitter and receiver 38. The remote control unit 37 also includes a data input device 39 and a monitor 40 for displaying data received from the patch 11. The data input 39 may be a keyboard for example and monitor 40 may be a data display screen of one of the known forms. Remote control unit 37 enables transmission of signals, which are preferably encrypted, to the internal radio transmitter and receiver 36 of patch 11 for such purposes as selecting a mode of operation of the patch and for programming or reprogramming the timing and duration of successive administrations of a bio-active agent. Monitor 40 displays information produced by data processor 34 such as readings of the concentration of a substance in a patient's body that are detected by the analysis unit 25. This allows medical personnel to control treatment of a patient without removal of the patch 11 from the patient or other manipulations at the actual patch and without necessarily being in proximity to the patient.

Remote control of the patch 11 can be useful in circumstances other than in medical treatment of an ill patient. For example, there is much concern in military operations about the possible use of chemical or biological weapons. Patches 11 containing one or more antitoxins, vaccines or the like can be fastened to the skin of soldiers and other persons who may be at risk but not be activated until use or imminent use of such weapons is detected. Upon detection of such a threat, military commanders may then immediately and simultaneously use remote control 37 to initiate administration of appropriate counter agents to all persons equipped with the patch.

Data processor 34 may be programmed to cause administration of a pre-determined amount of a drug or other agent at predetermined intervals following activation of the patch and the amount and interval can be changed by instructions transmitted by remote control 37 if necessary. The concentration of a therapeutic drug in the body diminishes following each administration as the drug is consumed by body processes. The rate at which the concentration of most particular drugs decreases is known to medical practitioners and dosage is repeated at intervals to maintain the concentration within a desired range. This can be a somewhat erratic process when the repeated doses require attention and efforts by the patient or medical personnel. The present invention provides for a more precise maintenance of the desired concentration in an automatic manner. In particular, data processor 34 can be programmed to provide an initial dosage of a drug or the like which brings the concentration up to or near the maximum value of the desired range and to provide a smaller dosage at appropriate intervals thereafter which restores the concentration to the initial value.

In particular, the following values can be entered into the memory of the data processor:
(t₀)=the time following activation of the patch at which the driver is to be energized to begin administration of the drug;
(N)=the initial dosage which is to be administered to the particular patient in order to achieve an initial concentration of the drug in the body;
(tₒₙ)=the period of time that the driver electrodes are to remain energized in order to deliver the initial dosage (N);
(V)=voltage to be applied to the driver electrodes in order to deliver the initial dosage (N) in time period (tₒₙ);
(1/n)=a fraction of the initial concentration by which the concentration is to be allowed do diminish before a replenishment dosage is administered. For example, (1/n) may be the half life of the initial dosage of the drug in the body in which case n=2;
(tₛₕᵤₜ)=the period of time that the driver electrodes are to be unenergized following each on period (tₒₙ). Time period (tₛₕᵤₜ) is the time required for the concentration of the drug in the body to diminish by fraction (1/n) of the initial concentration;
(t_{off})=the period of time following (t₀) after which the patch is to stop administering the drug.

At time t₀ following activation of the patch, the program signals voltage regulator 32 and switching circuit 33 to apply voltage V to the driver electrodes. After elapse of time tₒₙ the program signals the switching circuit to de-energize the driver electrodes. Subsequently, after elapse of time period tₛₕᵤₜ, the program signals the switching circuit to reapply voltage V to the driver electrodes for a time period equal to tₒₙ×(1/n) in order to administer the first replenishment dose. The program then continues to energize the driver electrodes with voltage V for cyclical time periods having a duration equal to tₒₙ×(1/n) and which are separated by time periods equal to tₛₕᵤₜ. The program terminates administration of the drug after time period t_{off} has elapsed.

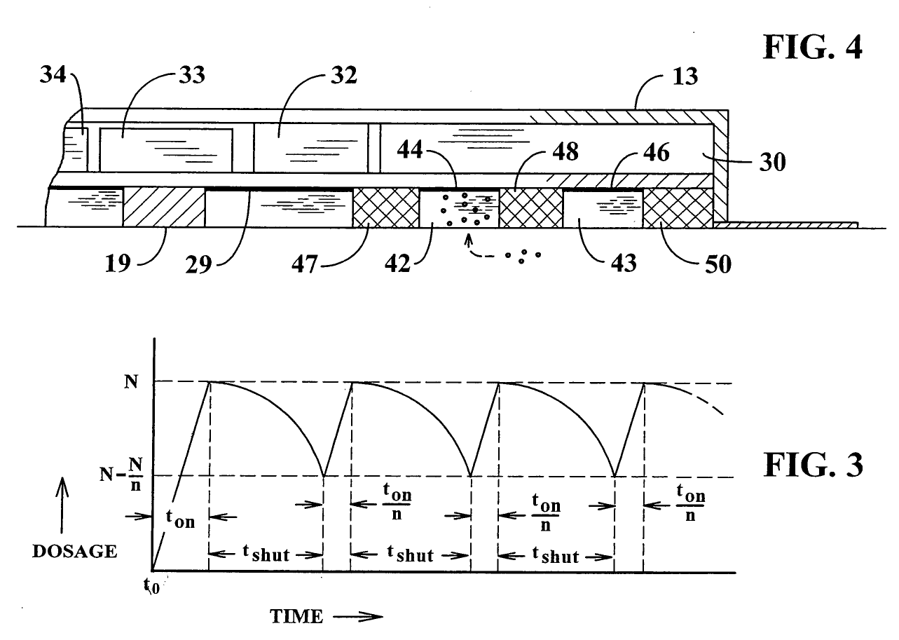

FIG. 3 graphically depicts the above described pattern of administration of a typical drug. For purposes of example FIG. 3 depicts the administration of testosterone which has a 12 minute metabolic half life in the human body and which is to be replenished each time that the concentration has declined to one half of the original value.

The patients need for some drugs may vary in a known cyclical fashion during the course of a day. Data processor 34 may be programmed to vary the dosage during the day or other time periods in the optimum manner.

The need for some other drugs or agents does not follow a predictable pattern of the kind described above. The need for such drugs may vary in a seemingly random manner dependent on the patient's activities, food consumption or other variables. The need for insulin by diabetic patients is a well known example. Referring again to FIGS. 1, 2 and 4, analysis unit 25 may be activated to monitor the concentration of a substance in a patient's body in a non-invasive manner. This enables variation of the timing and amount of successive dosages of one or more drugs to accommodate to an unpredictable need for a drug.

Analysis unit 25 operates by the reverse iontophoresis process in which an electrical current extracts interstitial fluid, including glucose for example, through the skin. The analysis unit 25 includes first and second spaced apart hydrogel fluid collection pads 42 and 43 respectively which are disposed at the underside of sector 24 of the patch in position to contact the skin. A flat electrode 44 is disposed against the upper surface of collection pad 42 and a similar electrode 46 is disposed against the upper surface of collection pad 43. In response to programmed instructions from data processor 34, switching circuit 33 applies positive voltage to electrode 44 and negative voltage to electrode 46 to create an electrical current in the underlying skin. This enhances porosity of the skin and causes negatively charged ions, such as glucose ions for example, to be drawn through the skin and into collection pad 42 by the current and the positive electrical charge on electrode 44. Negatively charged drug ions are drawn into the other collection pad 43 by a similar process.

An infrared source 47 directs infrared energy through collection pad 42 towards an infrared detector 48 which is situated between the two collection pads 42 and 43. Substances such as glucose absorb discrete infrared frequencies. The frequency absorption patterns for different particular substances, such as glucose, are known to the art and are unique to the particular substance. Thus the infrared intensity data produced by detector 48 for a series of specific infrared frequencies identifies the presence of a substance such as glucose in collection pad 42 and identifies the concentration of the substance in the pad. Detector 48 is of the type which outputs this data in digital form thereby enabling data processor 34 to analyze the detected data and to enable administration of a corrective dosage of an agent, such as insulin derivative, at one or more of the drug administration sectors 22, 23 and 24 in the manner previously described.

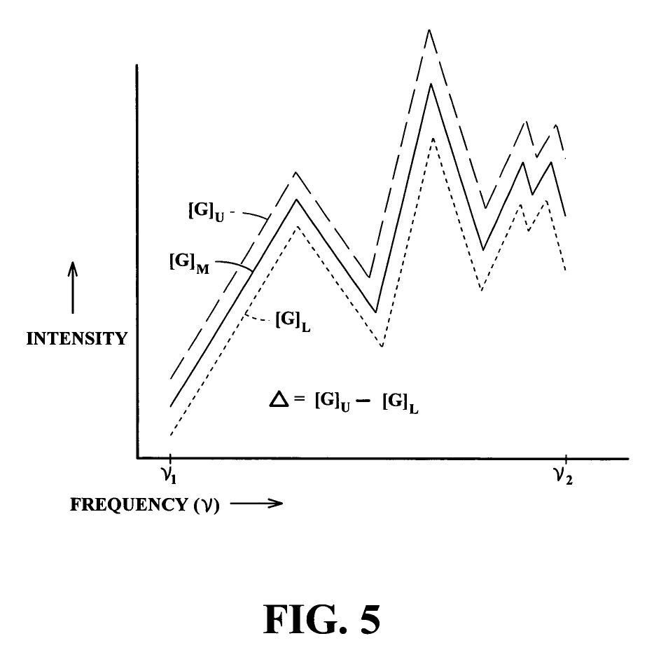

Referring jointly to FIGS. 4 and 5, data processor 34 may be programmed to sample a substance such as blood glucose “M” times per day starting at a specific hour (t_{M}) of the day. At time (t_{M}) the program signals switching circuit 33 to apply voltage to the analysis unit 25 thereby creating an electrical current in the underlying skin. This causes interstitial fluid to be drawn through the skin and into the analysis unit 25 by the reverse iontophoresis process. Ions which carry a positive charge, such as glucose ions, are drawn into collection pad 42 by the electrical current and the negative charge on the overlying electrode 44. Infrared source 47 directs infrared energy through the collection pad 42 and towards infrared detector 48. The infrared radiation includes the infrared absorption spectrum frequency range (v₁ to v₂) of the substance which is to be detected.

Prior to use of the patches with particular patients a series of “K” different known concentrations of the substance to be detected, such as glucose for example, are measured and their spectra [G]_{K} are stored in the permanent memory of data processor 34 in a vector (Qᵢ) whose successive elements represent detected infrared intensities and their corresponding frequencies. During use of the patch the program compares the detected infrared spectrum [G_{M}] of the glucose or other substance that is contained in each sampling of interstitial fluid with the stored vectors (Qᵢ) of intensities and frequencies for each concentration [G]_{K}. FIG. 5 is a diagrammatic depiction of a detected spectrum [G_{M}] which lies between two stored spectral values [G_{U}] and [G_{L}]. The upper spectrum [G_{U}] is the stored spectrum which is immediately above detected spectrum [G_{M}] and the lower spectrum [G_{L}] is the stored spectrum which is immediately below the detected spectrum. Therefore the difference (Δ) between the intensity levels of the successive spectra, such as [G_{U}] and [G_{L}], that are stored in the data processor memory determines the accuracy of the detected spectrum [G_{M}]. This difference can be made arbitrarily small to provide a desirable degree of accuracy by storing concentration spectra [G]_{K} which are arbitrarily closer to each other.

This form of programming enables determination of the concentration levels of glucose or other substances by the most basic fixed point computer operations instead of more complex floating point operations. This enables a very simple, inexpensive central processor to be employed.

The stored spectra [G]_{K} of glucose or another substance include the spectra of the maximum and minimum acceptable concentrations in the patient's body. If the detected concentration is above the maximum or below the minimum, the program initiates one or more modes of corrective action. In one mode of operation the program causes data processor 34 to signal radio receiver/transmitter 36 to transmit the concentration data to the remote radio receiver/transmitter 38 where medical personnel are alerted to the problem. Using remote control 37, the medical personnel may then return instructions to data processor 34 to cause administration of a corrective drug from one or more sectors of the patch 11 in the previously described manner. In another mode of operation, the program may cause the cyclical measurements of the concentration of a substance, such as glucose, to be stored in the random access memory of the data processor. Medical personnel may then use remote control 37 to access this information. In another mode of operation, the program may initiate administration of a corrective agent automatically when the detected concentration of a substance is outside of a desired range of concentrations.

Operations of the data processor 34 involving the analysis unit 25 have been described above primarily with reference to the monitoring of glucose in the body of a patient. The patch 11 can be adapted to monitoring other substances in the interstitial fluid of a patient's body by essentially similar techniques. The patch 11 can be adapted to monitor substances which are drawn towards the positive electrode 46 by reverse iontophoresis by providing another infrared source 50 which directs infrared towards detector 48 through the other collection pad 43.

The analysis unit 25 described above uses infrared spectrometry to monitor the concentration of a substance in interstitial fluid. A variety of other techniques are known which detect the concentration of a substance in a fluid. These other techniques may be used in the patch 11 in instances where the components for implementing the process are small enough to be contained in a transdermal patch.

Referring again to FIG. 1, a sizable period of time may elapse between manufacture of the patch 11 and the time that the patch is to be used. It is preferable that battery 30 be disconnected from the electronic components 32, 33, 34, 36 of the patch during this period of time to avoid premature operation of the patch and to avoid unnecessary draining of the battery. An activating switch 51 is provided to maintain the battery 30 in a disconnected state until such time as the patch is intentionally activated. While a simple on-off switch might be used for this purpose, it is preferable that the switch 51 have a specialized construction which blocks closing of the switch until an intentional action is taken to condition the patch 11 for operation. It is also preferable that the switch lock itself in the closed position when it is operated. This prevents accidental inactivation of the patch 11 during use.

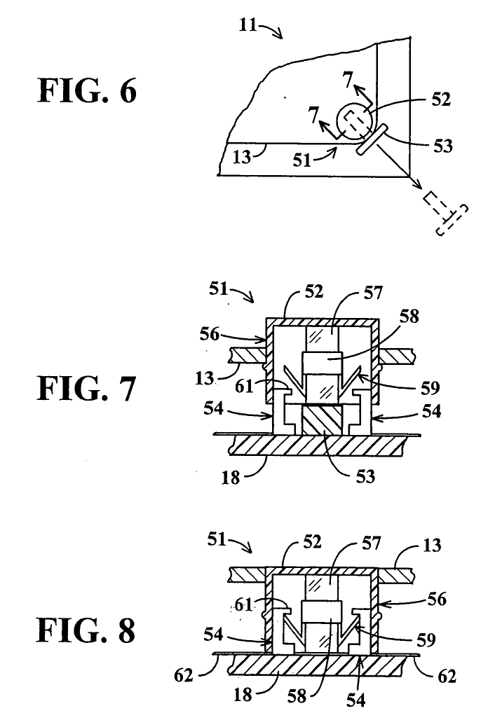

In particular, with reference to FIGS. 6, 7 and 8, the switch 51 of this example has a depressible switch button 52 which protrudes slightly from cover 13 at a corner of the cover when the switch is in the open condition. A removable plug 53 extends into cover 13 at that corner and prevents button 52 from being depressed until the plug is withdrawn from the cover. A spaced apart pair of metal fixed contacts 54 extend upward from circuit board 18 under button 52. Button 52, which is formed of non-conductive material, has a downward extending annular sleeve portion 56 which encircles the upper ends of fixed contacts 54 when the button is in its un-depressed condition. The button 52 also has a central rod portion 57 which extends downward between the upper ends of fixed contacts 54 when the button is in the un-depressed condition. A movable contact 58 is secured to rod portion 57 and has a pair of tangs 59 which extend outward and upward from opposite sides of the rod portion and which are formed of resilient metal.

The upper ends of fixed contacts 54 have small lips 61 which extend towards each other and which are positioned to deflect tangs 59 towards rod portion 57 temporarily as button 52 is traveled downward. Tangs 59 spring outward after passing between lips 61 and the lips then prevent the tangs and button 52 from being raised. Thus the switch 51 is locked at the closed position at which movable contact 58 forms an electrically conductive path between the fixed contacts 54. Separate conductors 62 extend from each fixed contact 54 to enable the battery to supply operating current to previously described electronic components of the patch when switch 51 is in the closed condition. At the closed position of the switch, sleeve 56 closes the opening in cover 13 that was created by withdrawal of plug 53

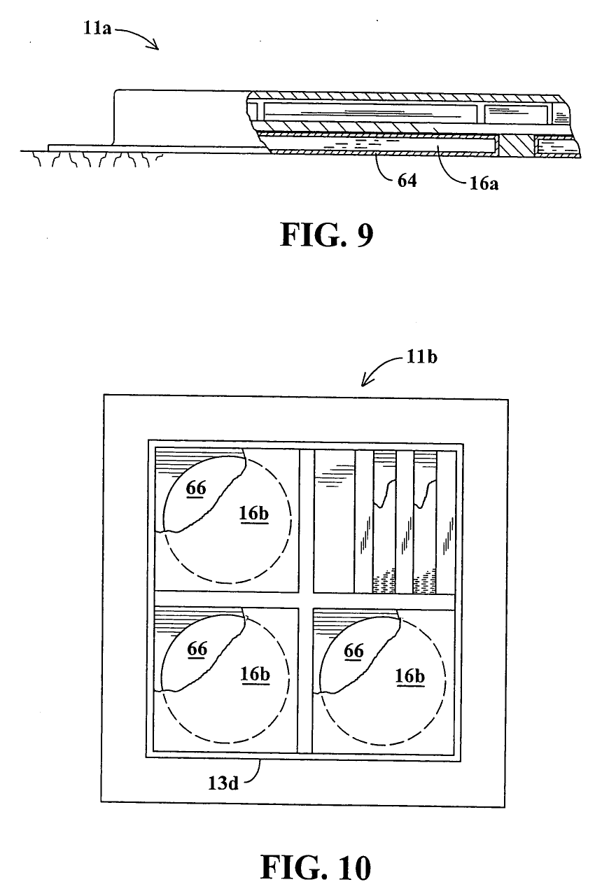

As previously described, the bio-active agent storage pads 16 are treated with additive to have a viscosity sufficient to retain the drug in the absence of an electrical current. FIG. 9 depicts an alternate embodiment in which storage pads 16a of the patch 11a need not be treated in this manner as they are each encased in a thin membrane 64 which is itself impermeable to the drug in the absence of an electrical current and which becomes permeable in the presence of current. The membranes 64 may be thin films of hydrogel material treated with a viscosity adjusting additive in the manner which has been previously described.

The above described embodiments of the invention have active drivers which rely on the iontophoresis process produced by subjecting the skin to an electrical current. Alternately the patch may make use of the process known as phonophoresis in which pulses of acoustic energy in the ultrasound range act to increase permeability of the outermost layer of the skin. Referring to FIG. 10, the driver electrodes of the previously described embodiments of the invention are replaced with small electrically operated ultrasound generators 66 which may be of known design. The ultrasound generators 66 are situated immediately above the hydrogel pads 16b. The patch 11b of FIG. 10 may otherwise be similar to the patch which has been previously described.

While the invention has been described with reference to certain specific embodiments for purposes of example, many other variations and modifications are possible and it is not intended to limit the scope of the invention except as defined by the following claims.

1. A transdermal patch for delivery of a bio-active agent into the skin of a living body which patch is fastenable to a surface of the skin, the patch containing at least one agent storage pad positioned to dispense agent into the skin and containing electrically operated driver means for causing delivery of the stored agent from the storage pad into the skin and containing a battery for supplying electrical current to the driver means, further including:
a programmable digital data processor controlling dispensing of said agent by said reservoir pad and driver means,
an analysis unit for sensing the concentration of a substance in the body, said analysis unit providing concentration signals to said digital data processor enabling dispensing of said agent into the skin when said concentration is outside of a particular range of concentrations
said programmable digital data processor and said analysis unit being contained within said patch.

2. The transdermal patch of claim 1 wherein said patch contains a plurality of said agent storage pads each storing a different agent, further including a plurality of said electrically operated driver means each being operative on a separate one of said plurality of agent storage pads in response to actuating signals from said data processor.

3. The transdermal patch of claim 2 further including a radio receiver contained by said patch for receiving programming signals for said digital data processor to enable actuation of selected ones of said electrically operated driver means in response to radio signals originating at a location which is spaced apart from said patch.

4. The transdermal patch of claim 1 wherein said driver means includes a first driver electrode disposed against said agent storage pad in electrical contact therewith at a first area of said skin and a second driver electrode positioned to be in electrical contact with a second area of said skin that is spaced apart from said first area.

5. The transdermal patch of claim 1 wherein said driver means includes an ultrasound generator disposed over said agent storage pad, said ultrasound generator being contained within said patch.

6. The transdermal patch of claim 1, said analysis unit having first and second spaced apart sensor electrodes within said patch which are positioned to establish an electrical current within a portion of the underlying skin to withdraw said substance through the skin, a collection pad within the patch which is positioned to receive the withdrawn substance, and a detector within the patch for detecting the concentration of said substance in said collection pad.

7. The transdermal patch of claim 6 wherein said detector comprises an infrared source at one side of said collection pad in position to direct infrared energy towards an infrared detector at an opposite side of said collection pad to detect the infrared absorption spectra of said substance in said collection pad including the intensity level of said infrared absorption spectra for analysis by said data processor.

8. The transdermal patch of claim 1 wherein said substance is glucose and wherein said bio-active agent is an insulin derivative drug.

9. The transdermal patch of claim 1 further including an internal radio transmitter for transmitting signals indicative of said concentration of said substance to a location which is spaced apart from said patch, said internal radio transmitter being contained within said patch.

10. The transdermal patch of claim 1 further including an internal radio transmitter and receiver contained within said patch, said internal radio transmitter and receiver being conditioned to transmit signals indicative of said concentration of said substance and to receive programming signals for said data processor, further including a remote radio transmitter and receiver situated apart from said patch and being conditioned to transmit said programming signals to said internal radio transmitter and receiver and to receive said signals indicative of said concentration of said substance.

11. The transdermal patch of claim 1 wherein said data processor is programmed to deliver repetitive doses of said agent at a repetition rate which corresponds to the rate at which the concentration of said agent in the body diminishes to a particular value.

12. The transdermal patch of claim 1, said data processor being programmed to vary the dosage of said agent during a period of time to conform to variations of the rate at which the agent is needed by the body during the period of time.

13. The transdermal patch of claim 1 wherein said agent storage pad is formed of a material which is impermeable by said bio-active agent in the absence of an electrical current and which becomes permeable by said bio-active agent when subjected to an electrical current.

14. The transdermal patch of claim 1 wherein said agent storage pad is bounded by a membrane, said membrane being a material which is permeable by said bio-active agent when subjected to an electrical current and which is impermeable by the bio-active agent in the absence of the electrical current, wherein said electrically operated driver means applies current to said membrane to enable release of the stored agent to the skin.

15. The transdermal patch of claim 1 wherein said agent storage pad is bounded by a membrane, said membrane being a material which is permeable when subjected to ultrasonic sound and which is impermeable in the absence of the ultrasonic sound, wherein said electrically actuated driver generates ultrasonic sound to enable release of the stored agent to the skin.

16. A transdermal patch for delivery of a bio-active agent into the skin of a living body which patch is fastenable to a surface of the skin, the patch containing at least one agent storage pad positioned to dispense agent into the skin and containing electrically operated driver means for causing delivery of the stored agent from the storage pad into the skin and containing a battery for supplying electrical current to the driver means, further including:
a programmable digital data processor controlling dispensing of said agent by said reservoir pad and driver means, and
a radio receiver which inputs programming signals to said programmable digital data processor,
said programmable digital data processor and said radio receiver being contained within said patch.

17. The transdermal patch of claim 15 further including a remote radio transmitter for transmitting said programming signals to said radio receiver, said radio transmitter being at a location which is spaced apart from said patch.
